(19) United States
(12) Patent Application Publication
(10) Pub. No.: US 2020/0397718 A1
(43) Pub. Date: Dec. 24, 2020

(54) USE OF HYDROXYTYROSOL ACETATE IN IMPROVING FUNCTION OF AORTIC ENDOTHELIAL CELL

(71) Applicant: XI'AN JIAOTONG UNIVERSITY, Xi'an (CN)

(72) Inventors: JIAN-KANG LIU, Xi'an (CN); XU-YUN LIU, Xi'an (CN); XIAO-JIE WANG, Xi'an (CN); YUE XIONG, Xi'an (CN); LIN ZHAO, Xi'an (CN); CAI-YUE ZHU, Xi'an (CN)

(21) Appl. No.: 16/897,272

(22) Filed: Jun. 10, 2020

(30) Foreign Application Priority Data
Jun. 18, 2019 (CN) 201910526194.1

(51) Int. Cl. A61K 31/05

(52) U.S. Cl. CPC A61K 31/05

(57) ABSTRACT

The present disclosure discloses use of hydroxytyrosol acetate in preparation of a medicament for improving aortic endothelial cell function. The hydroxytyrosol acetate is capable of inhibiting an inflammation of aortic endothelial cells caused by a saturated fatty acid, reducing mRNA levels corresponding to matrix metalloproteinase-1 (MMP-1), intercellular adhesion molecule-1 (ICAM-1) and plasma plasminogen activator inhibitor-1 (PAI-1) in aortic endothelial cells and protecting mitochondria from being damaged by inflammation of aortic endothelial cells caused by a saturated fatty acid, preventing the occurrence and development of atherosclerosis by anti-inflammatory and protecting mitochondrial function.

### SEQUENCE LISTING

This application contains a Sequence Listing in computer readable form. The computer readable form is incorporated herein by reference.

### CROSS-REFERENCE TO RELATED APPLICATION

This application claims all benefits accruing under 35 U.S.C. § 119 from China Patent Application No. 201910526194.1, filed on Jun. 18, 2019 in the China National Intellectual Property Administration, the content of which is hereby incorporated by reference.

### FIELD

The present disclosure relates to the pharmaceutical field, and particularly relates to use of hydroxytyrosol acetate in improving aortic endothelial cell function.

### BACKGROUND

Statistical data released by the World Health Organization (WHO) in 2011 shows that deaths caused by cardiovascular diseases account for 31% of global deaths, second only to the total of other non-communicable diseases. It is expected that the mortality caused by cardiovascular diseases will be significantly higher than other diseases from 2008 to 2030, taking the lead position. The pathological basis of cardiovascular diseases and cerebrovascular diseases, such as myocardial infarction and cerebral infarction, is atherosclerosis.

Atherosclerosis, whose pathogenesis is very complex, is a chronic inflammatory response with plaques inside arteries, and accompanied by damages to vascular endothelial cells. The main factors leading to atherosclerosis are an unhealthy diet such as high-salt, high-fat, and high-energy diet, smoking, and metabolic risk factors, including diseases such as the “Three-High” symptom (hypertension, hyperglycemia, and hyperlipidemia) and obesity.

Although atherosclerosis may be treated with medicine or surgery, it is latent leading to a high lethality rate and a high disability rate. Therefore, prevention and early treatment of atherosclerosis are particularly favorable. Studies have revealed that natural active ingredients such as chlorogenic acid, “Xiongshao” (Ligusticum chuanxiong and Paeoniae rubra radix) and lignans have anti-atherosclerotic effects, and functional foods containing these ingredients are available on the market. It is of great significance and prospect to explore natural substances that are effective against cardiovascular diseases such as atherosclerosis.

Hydroxytyrosol acetate was discovered in 1999, and is a compound found naturally in olive oil. A content of hydroxytyrosol acetate in Spanish olive oil is 7 times higher than that in Blanca olive oil. Hydroxytyrosol acetate can be produced not only from most olive varieties, but also from black olives by natural fermentation. Hydroxytyrosol acetate can be used not only in cosmetics, but also to prevent cerebral ischemic stroke, liver and kidney diseases, and physical damage caused by muscle damage.

### SUMMARY

The present disclosure provides a pharmaceutical composition containing hydroxytyrosol acetate, a method for improving aortic endothelial cell function, and use of hydroxytyrosol acetate in preparation of a medicament for improving aortic endothelial cell function.

Hydroxytyrosol acetate can improve aortic endothelial cell function, significantly inhibit inflammatory response of human aortic endothelial cells induced by a saturated fatty acid, and increase production of membrane fusion protein of mitochondrial complex in vascular endothelial cells, preventing the occurrence and development of atherosclerosis by anti-inflammatory and protecting mitochondrial function.

Hydroxytyrosol acetate is capable of reducing mRNA levels corresponding to matrix metalloproteinase-1 (MMP-1), intercellular adhesion molecule-1 (ICAM-1) and/or plasma plasminogen activator inhibitor-1 (PAI-1) in aortic endothelial cells.

Hydroxytyrosol acetate is capable of reducing a mRNA level corresponding to interleukin-6 (IL-6) in aortic endothelial cells.

Hydroxytyrosol acetate is capable of protecting mitochondria from being damaged by inflammation of aortic endothelial cells caused by a saturated fatty acid.

Hydroxytyrosol acetate is capable of reducing a production of excessive reactive oxygen species in aortic endothelial cells and increasing an expression of mitochondrial membrane fusion-related protein Mfn2 in aortic endothelial cells.

The hydroxytyrosol acetate can be used in preparation of a pharmaceutical composition, such as a medicament or a nutritional supplement.

In an embodiment, the pharmaceutical composition is a medicament for improving aortic endothelial cell function.

In an embodiment, the pharmaceutical composition is a medicament for preventing or treating cardiovascular diseases.

In an embodiment, the pharmaceutical composition is a medicament for treating atherosclerosis.

In an embodiment, the pharmaceutical composition is a medicament or a nutritional supplement for preventing the occurrence of atherosclerosis.

The present disclosure provides the pharmaceutical composition that includes hydroxytyrosol acetate.

The pharmaceutical composition can further include a pharmaceutical acceptable diluent, an excipient, and/or a carrier.

The present disclosure provides a method for improving function of aortic endothelial cells, and the method includes contacting the cells with an effective amount of hydroxytyrosol acetate.

The present disclosure provides a method for inhibiting saturated fatty acid induced inflammation of aortic endothelial cells, and the method includes contacting the cells with an effective amount of hydroxytyrosol acetate.

The present disclosure provides a method for reducing mRNA levels corresponding to matrix metalloproteinase-1 (MMP-1), intercellular adhesion molecule-1 (ICAM-1) and/or plasma plasminogen activator inhibitor-1 (PAI-1) in aortic endothelial cells, and the method includes contacting the cells with an effective amount of hydroxytyrosol acetate.

The present disclosure provides a method for reducing a mRNA level corresponding to interleukin-6 (IL-6) in aortic endothelial cells, and the method includes contacting the cells with an effective amount of hydroxytyrosol acetate.

The present disclosure provides a method for protecting a mitochondrion from being damaged by saturated fatty acid induced inflammation of aortic endothelial cells, and the method includes contacting the cells with an effective amount of hydroxytyrosol acetate.

The present disclosure provides a method for increasing an expression of mitochondrial membrane fusion related protein Mfn2 in aortic endothelial cells, and the method includes contacting the cells with an effective amount of hydroxytyrosol acetate.

The present disclosure provides a method for reducing a production of excessive reactive oxygen species in aortic endothelial cells, and the method includes contacting the cells with an effective amount of hydroxytyrosol acetate.

The present disclosure provides a method for preventing or treating a cardiovascular disease, the method includes administering to a patient in need thereof a therapeutically effective amount of hydroxytyrosol acetate or the pharmaceutical composition.

The present disclosure provides a method for preventing atherosclerosis, the method includes administering to a patient in need thereof a therapeutically effective amount of hydroxytyrosol acetate or the pharmaceutical composition.

The present disclosure provides use of hydroxytyrosol acetate in preparation of a medicament for improving aortic endothelial cell function.

The present disclosure provides use hydroxytyrosol acetate in preparation of a medicament for preventing or treating a cardiovascular disease.

The present disclosure provides use hydroxytyrosol acetate in preparation of a medicament for treating atherosclerosis.

The present disclosure provides use hydroxytyrosol acetate in preparation of a medicament or a health care product for preventing the occurrence of atherosclerosis.

Hydroxytyrosol acetate has no toxic and side effects on human aortic endothelial cells. It is disclosed in the present disclosure for the first time that hydroxytyrosol acetate can significantly inhibit the inflammatory response of human aortic endothelial cells induced by a saturated fatty acid and increase the production of a mitochondrial membrane fusion-related protein in vascular endothelial cells, in vascular diseases such as atherosclerosis, that has a damage or inflammation of vascular endothelium. The hydroxytyrosol acetate prevents the occurrence and development of atherosclerosis by anti-inflammation and protecting the mitochondria.

The hydroxytyrosol acetate is capable of effectively reducing human aortic endothelial inflammation caused by a saturated fatty acid, for example, reducing mRNA levels corresponding to interleukin-6 (IL-6), metalloproteinase-1 (MMP-1), intercellular adhesion molecule-1 (ICAM-1) and/or plasma plasminogen activator inhibitor-1 (PAI-1). The hydroxytyrosol acetate is capable of effectively protecting the function of the mitochondria in human aortic endothelial cells from being damaged by a saturated fatty acid, for example, reducing the production of excessive reactive oxygen species in cells and increasing the mitochondrial membrane fusion-related protein Mfn2. The hydroxytyrosol acetate has a good application prospect in preventing the occurrence and development of vascular diseases.

### BRIEF DESCRIPTION OF THE DRAWINGS

Implementations are described by way of example only with reference to the attached figures.

FIG. 1 is a diagram showing non-toxic side effects of hydroxytyrosol acetate on human aortic endothelial cells, wherein the abscissa represents the concentration of hydroxytyrosol acetate, and the ordinate represents relative cell viability.

FIG. 2 is a diagram showing the inhibitory effect of hydroxytyrosol acetate in different concentrations on palmitic acid-induced inflammation of human aortic endothelial cells, wherein the abscissa represents the concentration of hydroxytyrosol acetate, and the ordinate represents mRNA level corresponding to IL-6, MMP-1, ICAM-1 and PAI-1.

FIG. 3A and FIG. 3B are graphs showing that hydroxytyrosol acetate is capable of reducing palmitic acid-induced excessive reactive oxygen species in human aortic endothelial cells. FIG. 3A shows micrographs and FIG. 3B is a diagram of results obtained from a microplate reader.

FIG. 4A and FIG. 4B are diagrams showing that hydroxytyrosol acetate is capable of up-regulating the expression of the palmitic acid-induced mitochondrial membrane fusion-related protein Mfn2 in human aortic endothelial cells. FIG. 4A shows a Western blot testing result, and FIG. 4B is a statistical diagram based on the Western blot testing result. The abscissa of FIG. 4B represents the protein name, and the ordinate of FIG. 4B represents relative protein expression level.

### DETAILED DESCRIPTION

A detailed description with the above drawings is made to further illustrate the present disclosure.

1. Experimental Materials

Hydroxytyrosol acetate was purchased from Shanghai Yuanye Biotechnology Co., Ltd., article number: 69039-02-7, HPLC purity ≥98%. TRIzol™ reagent was purchased from Invitrogen™. RNA reverse transcription kit and SYBR fluorescent dye were purchased from Takara Biotechnology (Dalian) Co., Ltd. RNA primers were ordered from and synthesized by Xi'an Qingkezexi Bio Co., Ltd.

2. Culture of Experimental Cells and Model Establishment

Human aortic endothelial cells (HAECs) were purchased from Shanghai Shanghai Bioleaf Biotech Co., Ltd. Palmitic acid (PA) was purchased from SIGMA Company. A palmitic acid solution having a concentration of 500 μM was prepared by dissolving palmitic acid in water. A cell culture incubator was adopted to culture the cells in a temperature-constant, humidified, sterile condition. The cells were cultured in wells of culture plates at an atmosphere of 95% air and 5% CO₂ at 37° C. in the incubator. The experiments were performed on different experimental groups of cells. The experiments were performed on three experimental groups of cells: (1) control group; (2) 500 μM palmitic acid treatment group; and (3) hydroxytyrosol acetate protection group.

3. Experimental Methods

(1) MTT (3-(4,5-Dimethylthiazol-2-yl)-2,5-diphenyltetrazolium bromide) assay

The HAECs that were previously cultured in a 12-well cell culture plate were divided into 4 different experimental groups and respectively applied with hydroxytyrosol acetate in different concentrations, 0 μM, 10 μM, 50 μM, and 100 μM, all followed with an 24-hour incubation. Then, each of the 4 groups of cells was further divided into two subgroups, one was applied with 500 μM palmitic acid, the other was not, all followed with another 24-hour incubation.

An MTT assay was performed to all groups of cells. The MTT assay was a laboratory test and standard colorimetric assay for measuring the activity of enzymes that reduced MTT to formazan, giving a purple colour. Yellow MTT was reduced to purple formazan in living cells. For each group, HAECs were washed with phosphate-buffered saline (PBS) once, applied with 0.5 mg/ml of MTT, and cultured in the incubator containing 95% air and 5% CO₂ at 37° C. for 4 hours. The cultured cells were then washed three times with PBS before adding dimethyl sulfoxide (DMSO) to turn the insoluble purple formazan product into a colored solution. The absorbance was measured at a wavelength of 490 nm in a spectrophotometer. The absorbance of this colored solution can be quantified by measuring at a certain wavelength (usually between 500 and 600 nm) by a spectrophotometer.

(2) Detection of mRNA Levels Corresponding to IL-6, MMP-1, ICAM-1 and PAI-1

The HAECs that were previously cultured in a 12-well cell culture plate were divided into 5 different experimental groups and respectively applied with hydroxytyrosol acetate in respective concentrations, 0 μM, 0 μM, 10 μM, 50 μM, and 100 μM, all followed with an 24-hour incubation. Then, the last 4 groups of cells were respectively applied with 500 μM palmitic acid, the first was not, all followed with another 24-hour incubation. The detecting of mRNA levels corresponding to interleukin-6 (IL-6), matrix metalloproteinase-1 (MMP-1), intercellular adhesion molecule-1 (ICAM-1) and plasma plasminogen activator inhibitor-1 (PAI-1) were respectively carried out on each group of cells by using reverse transcription RNA and real-time quantitative polymerase chain reaction (PCR), and the specific method is as follows:

1) RNA Extraction

The medium for cell culture in the wells was removed. 500 μL of TRIzol™ reagent was added to each well, and the culture plate was then shaken at room temperature for 5 minutes. Then the cells in the wells were collected and transferred to a 1.5 mL Eppendorf® (EP) tube. 200 μl (taking ⅕ of total volume of the substance in the EP tube) of chloroform was then added to the EP tube for extraction of protein. The samples were then vigorously stirred for 15 seconds, rested for 15 minutes at room temperature, and then centrifuged at a relative centrifugal force of 12,000 g for 10 minutes at 4° C. The upper aqueous phase of each sample was transferred to another new EP tube, to which isopropanol with a volume equal to the transferred upper aqueous phase was added and uniformly mixed with the transferred upper aqueous phase. The solution rested for 1 hour at −20° C., and then centrifuged at 12,000 g for 10 minutes at 4° C. The supernatant was discarded, and 1 mL of pre-cooled 75% ethanol was added to the RNA pellet and mixed by pipetting up and down. The solution was centrifuged at 12,000 g for 10 minutes at 4° C., and then the supernatant is discarded. The EP tube containing the RNA pellet was placed on a super-clean bench for 30 minutes to completely evaporate the ethanol, and the resultant was resuspended in 10 μL of DEPC-treated water to form a total RNA solution for the following reverse transcription. The concentration of the solution was measured by an ultraviolet spectrophotometer.

2) Reverse Transcription of RNA

For performing the reverse transcription, a solution with a total volume of 20 μl was prepared by mixing 2 μg of the extracted RNA, 0.5 μg of random primers, 4 μL of 5× Master Mix, and DEPC-treated water taking all the rest volume. The solution was incubated at 37° C. for 60 minutes to have the reverse transcription reaction to obtain cDNA, then inactivated at 80° C. for 15 seconds, and then stored at −20° C. for later use.

3) Real-Time Quantitative PCR (RT-PCR)

RT-PCR was performed by using the RNA reverse transcription kit and the SYBR fluorescent dye. A system with a total volume of 10 μL was prepared by mixing 1 μL of the obtained cDNA, 5 μL of 2×SYBP®Premix Ex Taq™ II, 0.5 μL of a mixture of forward primer and backward primer (10 μM), and sterilized water taking all the rest volume. The RT-PCR was performed according to instructions of the kit with a protocol as follows: unwinding at 95° C. for 10 minutes; performing PCR for 40 cycles, each of which was performed by sequentially subjecting the system at 95° C. for 30 seconds, at 55° C. for 30 seconds, and at 72° C. for 20 seconds; and finally observing and analyzing a dissociation curve performed by sequentially subjecting the system at 95° C. for 15 seconds, at 60° C. for 15 seconds, and at 95° C. for 15 seconds). β-actin was used as an internal reference, the primer sequences used in the experiment were as follows:

PCR primers for IL-6:
Forward primer:
(SEQ ID NO: 1)
5′-TTTTGTACTCATCTGCACAGC-3′
Backward primer:
(SEQ ID NO: 2)
5′-GGATTCAATGAGGAGACTTGC-3′
PCR primers for MMP-1:
Forward primer:
(SEQ ID NO: 3)
5′-ACGCCAGATTTGCCAAGAG-3′
Backward primer:
(SEQ ID NO: 4)
5′-TTGACCCTCAGAGACCTTGGT-3′
PCR primers for ICAM-1:
Forward primer:
(SEQ ID NO: 5)
5′-CTATGGCAACGACTCCTTCT-3′
Backward primer:
(SEQ ID NO: 6)
5′-CAGCGTCACCTTGGCTCTA-3′
PCR primers for PAI-1:
Forward primer:
(SEQ ID NO: 7)
5′-GCACAATCCCCCATCCTACG-3′
Backward primer:
(SEQ ID NO: 8)
5′-GGCTCTCTCCACCTCTGAAA-3′
PCR primers for 13-actin:
Forward primer:
(SEQ ID NO: 9)
5′-ATCATGTTTGAGACCTTCAA-3′
Backward primer:
(SEQ ID NO: 10)
5′-AGATGGGCACAGTGTGGGT-3′

(3) Detection of Reactive Oxygen Species (ROS)

The cultured HAECs were divided into 3 different experimental groups, the control (Ctrl) group, the PA group, and the hydroxytyrosol acetate+PA (HTac+PA) group. The cells of the HTac+PA group were applied with hydroxytyrosol acetate in a concentration of 100 μM followed with a 24-hour incubation. Then, the cells of both the PA group and the HTac+PA group were applied with 500 μM palmitic acid, all followed with another 24-hour incubation.

After treating the cells with hydroxytyrosol acetate and palmitic acid, and discarding the medium, the cells were rinsed once with PBS. 10 μmol/L H2DCF-DA working solution was added before incubating at 37° C. for 30 min. After discarding the medium, the cells were rinsed twice with PBS, and divided into two sub group, one was used for microscope photography, and the other was digested with trypsin and lysed followed by centrifugation at 13,000 g for 5 min at 4° C. The supernatant was collected and measured by a microplate reader.

(2) Protein Detection

The HAECs that were previously cultured in a 6-well cell culture plate were divided into 3 different experimental groups, the control (Ctrl) group, the PA group, and the hydroxytyrosol acetate+PA (HTac+PA) group. The cells of the HTac+PA group were applied with hydroxytyrosol acetate in a concentration of 100 μM followed with a 24-hour incubation. Then, the cells of both the PA group and the HTac+PA group were applied with 500 μM palmitic acid, all followed with another 24-hour incubation. The protein detection was respectively carried out on each group of cells as follows.

1) Protein Extraction

The medium for cell culture in the wells was removed. 150 μL of IP lysis buffer was added to each well of the culture plat. The cultured cells in the wells were scraped by using a cell scraper, collected and transferred to a 1.5 mL EP tube, and subjected to vibrating for 15 seconds and cooling in ice bath for 10 minutes. The vibrating and cooling were repeated three times, ensuring that the cells were ice bathed for at least 30 minutes. Then, the samples were centrifuged at 12,000 g for 10 minutes at 4° C. The supernatants were collected, and the proteins therein were quantified by bicinchoninic acid (BCA) assay, and normalized. Then, the supernatants were added with 5× loading buffer and mercaptoethanol, and boiled for 10 minutes to denature the proteins. The extracted proteins were stored at −80° C. for later use.

2) Western Blot

10 μg of the extracted proteins were subjected to gel electrophoresis with 10% acrylamide gel, and electrophoretic transferred onto a PVDF membrane, which were blocked, and incubated with a primary antibody at 4° C. overnight, free primary antibody was washed away. Then, the membrane was incubated with a secondary antibody at room temperature for 1 hour, and free secondary antibody was washed away. Target proteins were detected by chemiluminescence.

4. Statistical Analysis

The data obtained in the above-described experiments were expressed in the diagrams in form of mean±SEM (SEM is standard error of mean), and the data were analyzed by using One-way ANOVA analysis method with statistical significance p values of * meaning p<0.05, ** meaning p<0.01, *** meaning p<0.001.

5. No Toxic and Side Effects of Hydroxytyrosol Acetate on Human Aortic Endothelial Cells

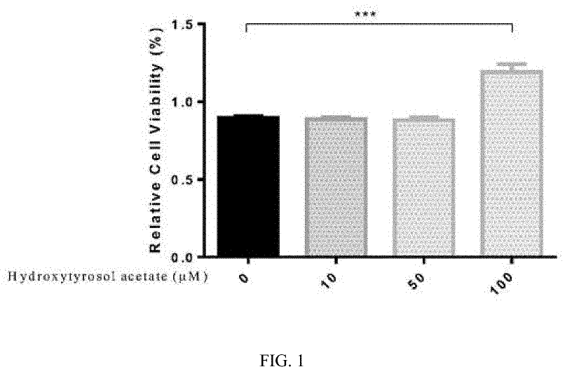

Referring to FIG. 1, the proliferation of human aortic endothelial cells (HAECs) was promoted in case where concentration of hydroxytyrosol acetate reaches 100 μM after applying hydroxytyrosol acetate to human endothelial cells for 24 h in the MTT assay.

6. Inhibitory Effect of Hydroxytyrosol Acetate on Palmitic Acid-Induced Inflammation of Human Blood Aortic Endothelial Cells

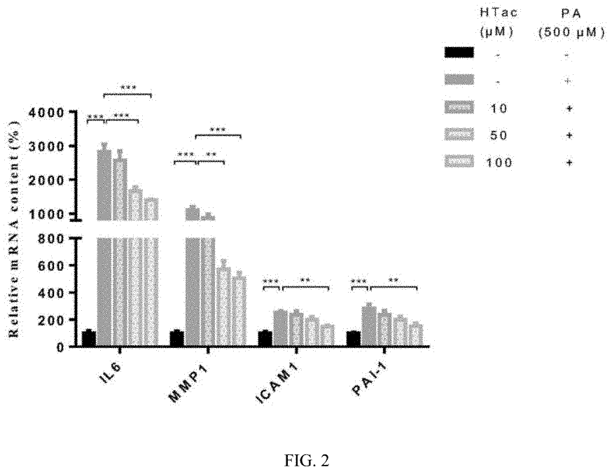

Referring to FIG. 2, hydroxytyrosol acetate significantly inhibited the inflammatory response induced by injury of the human aortic endothelial cells caused by the palmitic acid after applying hydroxytyrosol acetate to human aortic endothelial cells for 24 h followed by treatment with 500 μM of palmitic acid for 24 h. The inflammatory response of the human aortic endothelial cells can be induced by 500 μM palmitic acid, as indicated by the fact that the inflammatory cytokines IL-6, MMP-1, ICAM-1 and PAI-1 was significantly higher in the palmitic acid treated cells than that in control group. The mRNA levels corresponding to IL-6 and MMP-1 were increased for about 20 times and about 10 times, respectively, revealing that the inflammation level of the 500 μM palmitic acid treatment group is significantly increased. By applying hydroxytyrosol acetate with the concentration of 50 μM, a significant inhibition of the inflammatory response was generated. By applying hydroxytyrosol acetate with the concentration of 100 μM, the inhibitory effect is more obvious, suggesting the anti-inflammatory and anti-atherosclerotic effects of hydroxytyrosol acetate.

7. Inhibitory Effect of Hydroxytyrosol Acetate on Palmitic Acid-Induced Excessive Reactive Oxygen Species Generation in Human Aortic Endothelial Cells

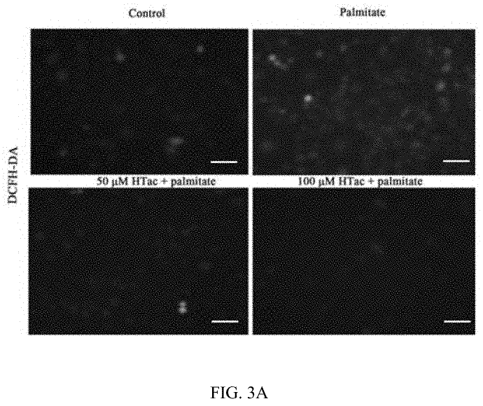

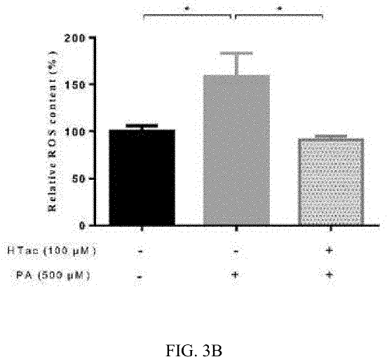

Oxidative stress is a negative effect produced by free radicals in the body and is considered to be an important factor leading to aging and disease. Palmitic acid can cause vascular endothelial cells to produce excessive reactive oxygen species (ROS), resulting in oxidative damage. FIG. 3A and FIG. 3B show that palmitic acid can induce human aortic endothelial cells to produce a large amount of reactive oxygen species ROS, while hydroxytyrosol acetate can significantly inhibit the production of excessive reactive oxygen species.

8. Upregulating Effect of Hydroxytyrosol Acetate on a Palmitic Acid-Induced Decrease of Expression of Mitochondrial Membrane Fusion-Related Protein Mfn2 in Human Aortic Endothelial Cells

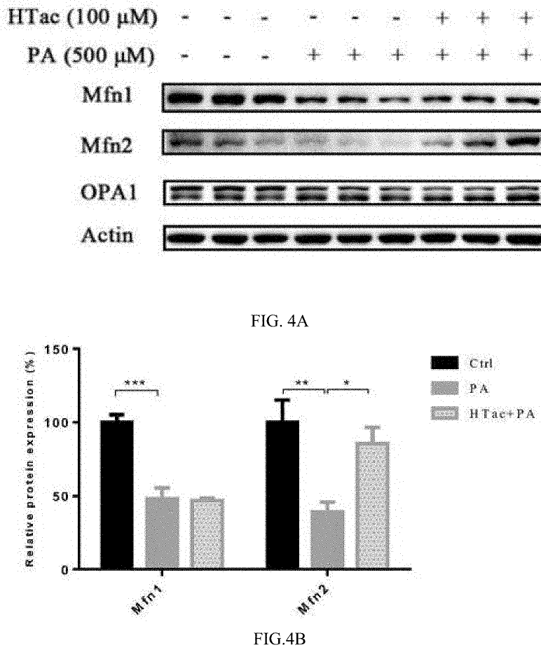

Mitochondrial fusion and division can not only change the shape of mitochondria and affect the function of mitochondria, but also affect the survival of cells. The mitochondrial fusion and division are precisely controlled by a variety of proteins. The proteins involved in mitochondrial fusion mainly include Mfn1, Mfn2 and OPAL FIG. 4A and FIG. 4B show that palmitic acid is capable of reducing the expression of mitochondrial membrane fusion-related proteins Mfn1, Mfn2 in human aortic endothelial cells, while hydroxytyrosol acetate is capable of significantly increasing the expression of Mfn2, thereby promoting cell fusion, and maintaining mitochondrial dynamic balance.

The above experimental results demonstrate that hydroxytyrosol acetate is capable of effectively inhibiting the inflammatory response and mitochondrial damage of human aortic endothelial cells induced by high fat, thereby improving the function of human aortic endothelial cells.

Hydroxytyrosol acetate can be used in preparing a pharmaceutical composition such as a medicament or a drag, for improving aortic endothelial cell function.

The pharmaceutical composition inhibits the inflammatory response of aortic endothelial cells caused by a saturated fatty acid.

The pharmaceutical composition reduces the mRNA levels of IL-6, ICAM-1 and PAI-1 in aortic endothelial cells.

The pharmaceutical composition protects mitochondria from being damaged by inflammation of aortic endothelial cells caused by a saturated fatty acid.

The pharmaceutical composition decreases the generation of excessive reactive oxygen species increases the expression of mitochondrial membrane fusion-related proteins Mfn2.

A dysfunction of endothelial cell is the initial characterization, the reason, and the basis of occurrence and development of atherosclerosis which is a chronic inflammatory response. The occurrence of the inflammatory response is an important cause of atherosclerosis. Meanwhile, it is reported that damage of the mitochondria may also be one important cause of atherosclerosis since the damage may induce an energy deficiency and function deterioration of endothelial cells. Hydroxytyrosol acetate exhibits excellent properties in protecting endothelial cells from inflammation and protecting mitochondria from being damaged in the endothelial cell damage test. Therefore, hydroxytyrosol acetate has a good prospect in prevention of cardiovascular diseases, such as atherosclerosis, that has an endothelial damage caused by high fat. Hydroxytyrosol acetate provides a new medical approach for treatment of cardiovascular diseases caused by imbalance of dietary.

Hydroxytyrosol acetate can be used in preparing a pharmaceutical composition for prevention or treatment of cardiovascular diseases.

Hydroxytyrosol acetate can be used in preparing a pharmaceutical composition for treatment of atherosclerosis.

Hydroxytyrosol acetate can be used in preparing a pharmaceutical composition or a nutritional supplement for prevention of atherosclerosis.

Finally, it is to be understood that the above-described embodiments are intended to illustrate rather than limit the present disclosure. Variations may be made to the embodiments without departing from the spirit of the present disclosure as claimed. Elements associated with any of the above embodiments are envisioned to be associated with any other embodiments. The above-described embodiments illustrate the scope of the present disclosure but do not restrict the scope of the present disclosure.

What is claimed is:

1. A pharmaceutical composition for ameliorating aortic endothelial cell function, the pharmaceutical composition comprising hydroxytyrosol acetate.

2. The pharmaceutical composition of claim 1, being medicine or a nutritional supplement.

3. The pharmaceutical composition of claim 1, further comprising a pharmaceutical acceptable diluent, an excipient, or a carrier.

4. The pharmaceutical composition of claim 1, wherein the hydroxytyrosol acetate is capable of inhibiting an inflammation of an aortic endothelial cell caused by a saturated fatty acid.

5. The pharmaceutical composition of claim 1, wherein the hydroxytyrosol acetate is capable of reducing mRNA levels corresponding to matrix metalloproteinase-1 (MMP-1), intercellular adhesion molecule-1 (ICAM-1) and/or plasma plasminogen activator inhibitor-1 (PAI-1) in an aortic endothelial cell.

6. The pharmaceutical composition of claim 1, wherein the hydroxytyrosol acetate is capable of reducing a mRNA level corresponding to interleukin 6 (IL-6) in an aortic endothelial cell.

7. The pharmaceutical composition of claim 1, wherein the hydroxytyrosol acetate is capable of protecting mitochondria from being damaged by inflammation of an aortic endothelial cell caused by a saturated fatty acid.

8. The pharmaceutical composition of claim 1, wherein the hydroxytyrosol acetate is capable of reducing a production of excessive reactive oxygen species in an aortic endothelial cell and increasing an expression of mitochondrial membrane fusion-related protein Mfn2 in the aortic endothelial cell.

9. A method for preventing or treating a cardiovascular disease, the method comprising administering to a patient in need thereof a therapeutically effective amount of the pharmaceutical composition of claim 1.

10. A method for preventing atherosclerosis, the method comprising administering to a patient in need thereof a therapeutically effective amount of the pharmaceutical composition of claim 1.

11. A method for improving function of an aortic endothelial cell, the method comprising contacting the cell with an effective amount of hydroxytyrosol acetate.

12. The method of claim 11, wherein the hydroxytyrosol acetate inhibits saturated fatty acid induced inflammation of an aortic endothelial cell.

13. The method of claim 11, wherein the hydroxytyrosol acetate reduces a mRNA level corresponding to one or more of matrix metalloproteinase-1 (MMP-1), intercellular adhesion molecule-1 (ICAM-1) and plasma plasminogen activator inhibitor-1 (PAI-1) in an aortic endothelial cell.

14. The method of claim 11, wherein the hydroxytyrosol acetate reduces a mRNA level corresponding to interleukin-6 (IL-6) in an aortic endothelial cell.

15. The method of claim 11, wherein the hydroxytyrosol acetate protects a mitochondrion from being damaged by saturated fatty acid induced inflammation of an aortic endothelial cell.

16. The method of claim 11, wherein the hydroxytyrosol acetate increases an expression of mitochondrial membrane fusion related protein Mfn2 in an aortic endothelial cell.

17. The method of claim 11, wherein the hydroxytyrosol acetate reduces a production of excessive reactive oxygen species in an aortic endothelial cell.

18. Use of hydroxytyrosol acetate in preparation of a medicament for improving aortic endothelial cell function.

19. The use of claim 18, wherein the medicament is for preventing or treating a cardiovascular disease.

20. The use of claim 18, wherein the medicament is for preventing or treating atherosclerosis.


SEQUENCE LISTING (10 sequences)

SEQ ID NO: 1 - DNA, 21 bp, Artificial Sequence
  Forward primer for IL-6
ttttgtactcatctgcacagc

SEQ ID NO: 2 - DNA, 21 bp, Artificial Sequence
  Backward primer for IL-6
ggattcaatgaggagacttgc

SEQ ID NO: 3 - DNA, 19 bp, Artificial Sequence
  Forward primer for MMP-1
acgccagatttgccaagag

SEQ ID NO: 4 - DNA, 21 bp, Artificial Sequence
  Backward primer for MMP-1
ttgaccctcagagaccttggt

SEQ ID NO: 5 - DNA, 20 bp, Artificial Sequence
  Forward primer for ICAM-1
ctatggcaacgactccttct

SEQ ID NO: 6 - DNA, 19 bp, Artificial Sequence
  Backward primer for ICAM-1
cagcgtcaccttggctcta

SEQ ID NO: 7 - DNA, 20 bp, Artificial Sequence
  Forward primer for PAI-1
gcacaatcccccatcctacg

SEQ ID NO: 8 - DNA, 20 bp, Artificial Sequence
  Backward primer for PAI-1
ggctctctccacctctgaaa

SEQ ID NO: 9 - DNA, 20 bp, Artificial Sequence
  Forward primer for beta-actin
atcatgtttgagaccttcaa

SEQ ID NO: 10 - DNA, 19 bp, Artificial Sequence
  Backward primer for beta-actin
agatgggcacagtgtgggt